(19) United States
(12) Patent Application Publication
(10) Pub. No.: US 2013/0203990 A1
(43) Pub. Date: Aug. 8, 2013

(54) PROCESS FOR THE PREPARATION OF IMATINIB MESYLATE

(71) Applicants: Rajasekhar Kadaboina, Hyderabad (IN); Munaswamy Sekhar Nariyam, Hyderabad (IN); Raghupati Rama Subrahmanyam Vinjamuri, Hyderabad (IN); Shravan Kumar Komati, Nalgonda (IN); Srinivas Benda, Nalgonda (IN); Ramaseshagiri Rao Pulla, Hyderabad (IN); Dharma Jagannadha Rao Velaga, Visakhapatnam (IN)

(72) Inventors: Rajasekhar Kadaboina, Hyderabad (IN); Munaswamy Sekhar Nariyam, Hyderabad (IN); Raghupati Rama Subrahmanyam Vinjamuri, Hyderabad (IN); Shravan Kumar Komati, Nalgonda (IN); Srinivas Benda, Nalgonda (IN); Ramaseshagiri Rao Pulla, Hyderabad (IN); Dharma Jagannadha Rao Velaga, Visakhapatnam (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/812,722

(22) PCT Filed: Jul. 28, 2011
(86) PCT No.: PCT/US2011/045655

§ 371 Date: Mar. 5, 2013

Related U.S. Application Data
(60) Provisional application No. 61/389,847, filed on Oct. 5, 2010.

(30) Foreign Application Priority Data
Jul. 29, 2010 (IN) 2161/CHE/2010

(51) Int. Cl. C07D 401/14

(52) U.S. Cl. CPC C07D 401/14
USPC 544/295

(57) ABSTRACT

The present application relates to process for the preparation of imatinib mesylate. This application also relates to the processes for preparation of alpha crystalline form of imatinib mesylate.

### INTRODUCTION

The present application relates to process for the preparation of imatinib mesylate. This application also relates to the processes for preparation of alpha crystalline form of imatinib mesylate.

Imatinib mesylate has a chemical name 4-[(4-methyl-1-piperazinyl)methyl]-N-[4-methyl-3-[[4-(3-pyridinyl)-2-pyrimidinyl]amino]-phenyl]benzamide methane sulfonate and can be represented by structural Formula I.

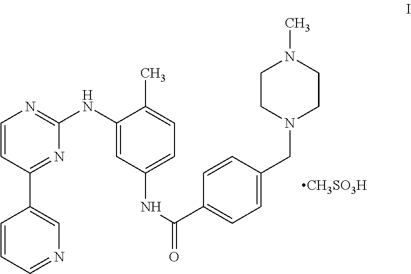

Imatinib is a protein tyrosine kinase inhibitor and is available in products sold using the trademark GLEEVEC® in the form of tablets containing imatinib mesylate equivalent to 100 mg or 400 mg of imatinib free base.

Zimmermann et al., in U.S. Pat. No. 5,521,184 describes the preparation of imatinib and its use as an anti-tumor agent. The patent describes the process for preparation of imatinib free base that involves reacting N-(5-amino-2-methylphenyl)-4-(3-pyridyl)-2-pyridimine amine with 4-(4-methylpiperazinomethyl)-benzoyl chloride in the presence of pyridine under nitrogen atmosphere for about 23 hours. The crude product obtained is made into slurry with solvent mixture of dichloromethane/methanol (95:5) and filtered to provide imatinib base.

Kankan et al., in U.S. Pat. No. 7,638,627 describes process for the preparation of imatinib or its hydrohalide salt which involves reacting N-(2-methyl-5-aminophenyl-4-(3-pyridyl)-2-pyridimine amine with 4-(4-methyl-piperazinomethyl)benzoyl chloride hydrohalide salt in the presence of an inert organic solvent for a prolonged duration so as to yield imatinib hydrohalide salt.

Xing et al., in U.S. Pat. No. 7,550,591 describes the process for the preparation of imatinib which involves reacting N-(5-amino-2-methylphenyl)-4-(3-pyridyl)-2-pyridimine amine with 4-(4-methyl-piperazine-1-ylmethyl)-benzoic acid using a carboxylic acid coupling agent. Further, it also discloses a process for the purification of imatinib base that involves dissolving crude imatinib in a first crystallization solvent, which can include a solvent mixture; evaporating at least a portion of the first crystallization solvent adding a second crystallization solvent to produce crystallization; cooling to precipitate imatinib and isolating the purified product; wherein the crystallization solvent system can include methanol, ethanol, 1-propanol, 2-propanol, dichloromethane, chloroform or a mixture thereof, which results in imatinib base containing 0.01% of N-(5-amino-2-methylphenyl)-4-(3-pyridyl)-2-pyridimine amine (by HPLC).

International Application Publication No. WO2008/136010 A1 describes a process for the preparation of highly pure imatinib which involves condensation of N-(5-amino-2-methylphenyl)-4-(3-pyridyl)-2-pyridimine amine and 4-(4-methyl-piperazinomethyl)benzoyl chloride dihydrochloride in the presence of potassium hydroxide and chloroform. The work-up process involves adding aqueous 30% solution of potassium hydroxide, separating the organic layer, suspending the organic layer in water, adjusting the pH to 3-4 using hydrochloric acid, separating the aqueous layer, adjusting the pH to 9-12 using sodium hydroxide and extracting the product with chloroform, precipitating the product by adding ethyl acetate, followed by isolating the product having a purity of 99.5%.

International Application Publication No. WO2008/117298 A1 describes a process for the preparation of imatinib base, which involves reacting a suspension of 4-[(4-methyl-piperazin-1-yl)methyl])benzoyl chloride dihydrochloride in 2-propanol and anhydrous potassium carbonate with N-(5-amino-2-methylphenyl)-4-(3-pyridyl)-2-pyridimine amine. International Application Publication No. WO2009/060463 A1 describes a process for the preparation of imatinib base using 4-(4-methylpiperazinomethyl)benzoic acid as the starting material and a process for crystallization of imatinib base from acetone to yield a product having a purity of 99.9% by HPLC.

Adin et al., in US 2006/0223817 A1 describes a process for preparing the crystalline imatinib base form I, the process comprising: dissolving imatinib base in a solvent and heating; allowing the solution to cool sufficiently to produce crystals of imatinib base form I; isolating the crystals; and optionally washing and drying the crystals; wherein the solvent comprises toluene, chloroform, dichloromethane, methyl ethyl ketone (MEK), methyl isobutyl ketone (MIBK), cyclohexanone, 4-methylcyclohexanone, ethyl acetate, methanol, isopropyl alcohol, 1-butanol or a mixture thereof. Loieseleur et al., in US 2007/0293504 A1 describes a process for the preparation of imatinib by using an intermediate of the formula:

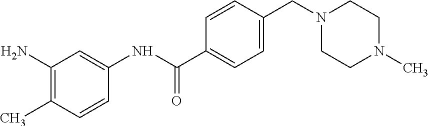 [SMILES: Cc1ccc(NC(=O)c2ccc(CN3CCN(C)CC3)cc2)cc1N]

According to the disclosure, the method of US '504 doesn't involve the use of expensive coupling reagents and has higher throughput combined with less steps than in the prior art results, which in significantly lower production costs.

Journal of Medicinal Chemistry 2005, 48, 249-255 discloses a process which involves reacting 4-chloro-methyl-N-[4-methyl-3-(4-pyridin-3-yl-pyridimin-2-yl-amino)phenyl]benzamide with N-methyl piperazine in acetonitrile to give imatinib which is further recrystallized from acetonitrile to provide creamy white crystals of imatinib base.

Despite existence of large number processes disclosed in the above mentioned prior art, there still exits a need for an improved process of preparation and purification of imatinib base and its pharmaceutically acceptable salt. The processes disclosed are not suitable and amenable to scale up due to one or more reasons. For example, the use of pyridine, long time durations for the reaction, formation of impurities, low yields, or the presence of mutagenic impurities/intermediates in the end product obtained. A process that involves the use of less hazardous and environmentally friendly reagents, solvents, and results in the imatinib base with significantly reduced quantities or is even free from potential process related impurities is needed.

The presence of mutagenic intermediates/impurities in the end product may be analyzed by Ames test (a specific test for mutagenicity; performed according to the OECD Guideline for Testing of Chemicals, 471: Bacterial Reverse Mutation Test, Adopted Jul. 21, 1997).

Zimmermann et al., in U.S. Pat. No. 6,894,051 describe two crystalline forms of imatinib mesylate, the alpha-form and the beta-form and processes for their preparation. The process for preparing crystalline alpha-form of imatinib mesylate involves the use of ethanol as the solvent, and involves several process steps.

International Application Publication No. WO2005/077933 A1 describes alpha 2-crystalline form of imatinib mesylate and process for the preparation of alpha 2-crystalline form of imatinib mesylate, which involves suspending imatinib base in 2-propanol, adding methanesulfonic acid, refluxing the reaction mixture, cooling, and filtering to yield the alpha 2-form. Further, it also describes another process which involves suspending the beta-form of imatinib mesylate in water and organic solvents like methanol, isopropyl ether, toluene, cyclohexane, or isopropyl alcohol; distilling off water azeotropically, cooling the solution, and filtering the reaction mass to afford alpha 2-form of imatinib mesylate.

Adin et al., in U.S 2006/0223816 A1 describe a stable, free-flowing imatinib mesylate alpha-form, which is substantially free of the beta-form. This application describes a process, which comprises:

a) heating a mixture of imatinib base and an organic solvent which includes methyl ethyl ketone, methyl isobutyl ketone, cyclohexanone, 4-methylcyclohexanone, acetonitrile and mixtures thereof, to dissolve at least a portion of imatinib base;

b) seeding with crystals of imatinib mesylate alpha-form;

c) gradually adding to the imatinib base solution a solution of methanesulfonic acid in the organic solvent;

d) allowing the mixture to cool, to precipitate crystals of imatinib mesylate in substantially pure form;

e) Isolating the precipitated crystals.

Szczepek et al., in US 2007/0197545 A1 describe a process for the preparation of alpha-crystal form of imatinib mesylate, which involves the use of not more than 0.99 equivalents of methanesulfonic acid per equivalent of imatinib in a solvent chosen from the group consisting of a C₂-C₆ aliphatic alcohol or mixtures thereof, optionally with the addition of C₁-C₄ aliphatic alcohol. The '545 application also discloses a process involving the use of solvents chosen from the group consisting of esters of lower carboxylic acids or C₁-C₄ aliphatic alcohols.

International Application Publication No. WO 2006/048890 A1 describes a non-needle shaped alpha-crystalline form of imatinib mesylate and a process for its preparation, which includes subjecting a solution of imatinib mesylate in a suitable solvent (which may be a polar protic or aprotic solvent, a non-polar solvent, water or a mixture thereof) to agitated thin film drying under atmospheric pressure and/or under vacuum.

International Application Publication No. WO 2007/136510 A2 describes processes for preparing crystalline imatinib mesylate of form alpha comprising providing a solution of imatinib mesylate in ethylene glycol dimethyl ether, and admixing the solution with tert-butyl methyl ether to form a suspension comprising said crystalline form or slurrying other crystalline forms of imatinib mesylate in solvents chosen from ethyl acetate, 1-propanol, acetone, or mixtures thereof.

Pathi et al., in US 2007/0265288 A1 describe a method of preparing crystalline imatinib mesylate alpha-form which involves suspending or dissolving imatinib base in a solvent chosen from 2-propanol, 1-propanol, 1-butanol, t-butanol, or methyl isobutyl ketone; adding methanesulfonic acid at a controlled rate and at predetermined temperature to the suspension or solution formed in step a; heating the mixture formed to at least about 70° C. to about at least 80° C. for a sufficient amount of time to induce the formation of alpha crystal form; and cooling the mixture to ambient temperature and isolating the crystalline alpha form.

Alexandr Jegorov et al., in US 2009/0264438 A1 describe processes for the preparation of α-crystalline form of imatinib mesylate:

a) by crystallizing imatinib mesylate from a solution of imatinib mesylate in a solvent chosen from the group consisting of: 1,2-propylene carbonate, a mixture of 1-propanol and acetic acid, or mixtures thereof or

b) by providing a solution of imatinib mesylate in ethylene glycol dimethyl ether, and admixing the solution with tert-butyl methyl ether to form a suspension comprising of the said crystalline form or

c) by slurrying imatinib mesylate chosen from a group consisting of: form IX, form VIII, or mixtures thereof, in a solvent chosen from the group consisting of: ethyl acetate, acetone, or mixtures thereof.

The above-mentioned documents collectively disclose diverse processes for the preparation of alpha-crystalline form of imatinib mesylate. Still there exists a need for a simple process for the preparation of alpha-crystalline form of imatinib mesylate resulting in high yield, purity, and residual solvents as per the ICH limit.

### SUMMARY

The present application relates to process for the preparation of imatinib mesylate. This application also relates to the processes for preparation of alpha crystalline form of imatinib mesylate.

In an embodiment, the present application provides a process for the preparing imatinib or its salt thereof comprising:

a) reacting N-(5-amino-2-methylphenyl)-4-(3-pyridyl)-pyrimidine-amine of formula II:

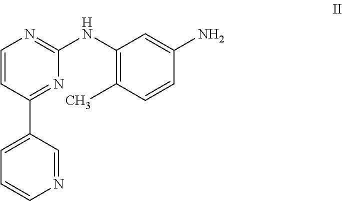 [SMILES: Cc1ccc(N)cc1Nc1nccc(-c2cccnc2)n1]

with 4-((4-methylpiperazin-1-yl)methyl)benzoyl chloride dihydrochloride of the formula III:

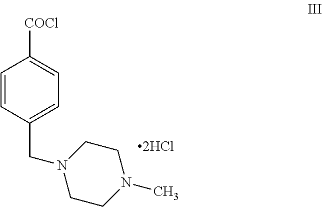 [SMILES: CN1CCN(Cc2ccc(C(=O)Cl)cc2)CC1]

in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof;

b) adjusting the reaction mass pH to 10-12 using aqueous sodium hydroxide;

c) extracting the reaction mass with dichloromethane;

d) adding water to the dichloromethane layer and adjusting the pH to 4-5 using an acid chosen from acetic acid, hydrochloric acid, oxalic acid, or citric acid;

e) separating the aqueous layer and washing with dichloromethane;

f) adding dimethylformamide and acetonitrile to the aqueous layer and adjusting the pH to 10-12 using aqueous sodium hydroxide;

g) isolating imatinib base of formula IV; and

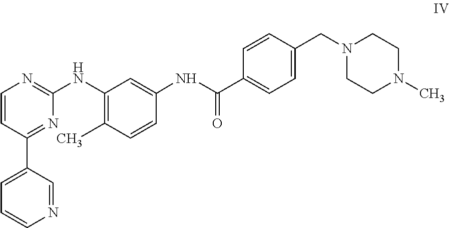 [SMILES: Cc1ccc(NC(=O)c2ccc(CN3CCN(C)CC3)cc2)cc1Nc1nccc(-c2cccnc2)n1]

h) optionally converting imatinib base to its mesylate salt of formula I.

### BRIEF DESCRIPTION OF THE DRAWINGS

FIG. 1 is an illustrative X-ray powder diffraction pattern of alpha-crystalline form of imatinib mesylate prepared by Example 3.

FIG. 2 is an illustrative X-ray powder diffraction pattern of alpha-crystalline form of imatinib mesylate prepared by Example 4.

FIG. 3 is an illustrative X-ray powder diffraction pattern of alpha-crystalline form of imatinib mesylate prepared by Example 7.

### DETAILED DESCRIPTION

The present application relates to process for the preparation of imatinib mesylate. This application also relates to the processes for preparation of the alpha crystalline form of imatinib mesylate.

In an embodiment, the present application provides a process for the preparing imatinib or its salt thereof comprising:

a) reacting N-(5-amino-2-methylphenyl)-4-(3-pyridyl)-pyrimidine-amine of formula II:

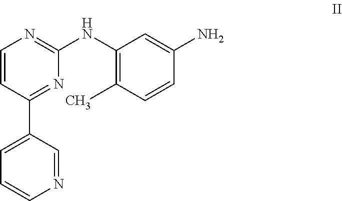 [SMILES: Cc1ccc(N)cc1Nc1nccc(-c2cccnc2)n1]

with 4-((4-methylpiperazin-1-yl)methyl)benzoyl chloride dihydrochloride of the formula III:

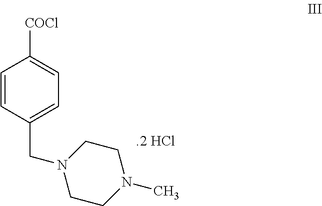 [SMILES: CN1CCN(Cc2ccc(C(=O)Cl)cc2)CC1]

in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof;

b) adjusting the reaction mass pH to 10-12 using aqueous sodium hydroxide;

c) extracting the reaction mass with dichloromethane;

d) adding water to the dichloromethane layer and adjusting the pH to 4-5 using an acid chosen from acetic acid, hydrochloric acid, oxalic acid, or citric acid;

e) separating the aqueous layer and washing with dichloromethane;

f) adding dimethylformamide and acetonitrile to the aqueous layer and adjusting the pH to 10-12 using aqueous sodium hydroxide;

g) isolating imatinib base of formula IV; and

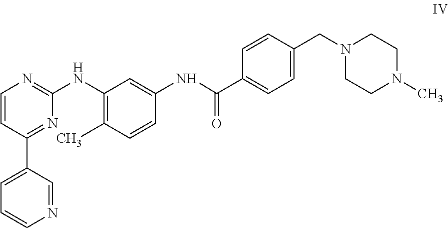 [SMILES: Cc1ccc(NC(=O)c2ccc(CN3CCN(C)CC3)cc2)cc1Nc1nccc(-c2cccnc2)n1]

h) optionally converting imatinib base to its mesylate salt of formula I.

The individual steps of the process are detailed below. Step a) involves reacting N-(5-amino-2-methylphenyl)-4-(3-pyridyl)-pyrimidine-amine of formula II with 4-((4-methylpiperazin-1-yl)methyl)benzoyl chloride dihydrochloride of the formula III in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof. The compounds of Formula II and III may be obtained by methods known in the art. The amount of base used may range from about 1 to about 10 molar equivalents, per molar equivalent of the compound of formula II. In an embodiment, about 6 to about 8 moles of N-methylmorpholine per molar equivalent of the compound of formula II is used as the base. The amount of compound of formula III used may range from about 1 to about 1.5 molar equivalents, per molar equivalent of the compound of formula II. The above reaction may be carried at temperatures from about −5° C. to about 35° C. In an embodiment, addition of the compound of the formula II to the compound of formula III in the above reaction is carried out at about −5° C. to about 5° C. and reaction mixture is maintained at temperatures from about −5° C. to about 5° C. for the further reaction. The use of base disclosed in the process of the present disclosure and carrying out the reaction at low temperatures results in the complete conversion of the starting material compound of the Formula II to imatinib base of formula IV.

Step b) involves adjusting the reaction mass pH to 10-12 using aqueous sodium hydroxide. After completion of the reaction, aqueous sodium hydroxide solution is added to the reaction mass for adjusting the pH about 10-12. In one embodiment, 10% aqueous sodium hydroxide solution is used.

Step c) involves extracting the reaction mass with dichloromethane. The reaction mass obtained in step b) is combined with dichloromethane, stirred, and separated to extract the product into dichloromethane. Optionally, the extraction process may be repeated to ensure complete transfer of the product and the extracts may be combined.

Step d) involves adding water to the dichloromethane layer and adjusting the pH to about 4-5 using an acid chosen from acetic acid, hydrochloric acid, oxalic acid, or citric acid. The dichloromethane layers obtained in step c) is combined with water and the pH of reaction mixture is adjusted to about 4-5 using the acid chosen. In an embodiment, 20% acetic acid is used for adjusting the pH of the reaction mass.

Step e) involves separating the aqueous layer and washing with dichloromethane. The biphasic reaction mixture obtained in step e) is subjected to separation of the layers and the aqueous layer is combined with dichloromethane, stirred and the phases separated for washing. Washing the reaction mixture at acidic pH (pH about 4 to 5) with dichloromethane or other suitable solvents removes the unreacted compound of formula II, if any, from the reaction mixture. Optionally, the process of washing the reaction mixture with dichloromethane may be repeated and the aqueous layer is taken forward for the next step of work-up.

Step f) involves adding dimethylformamide and acetonitrile to the aqueous layer and adjusting the pH of about 10-12 using aqueous sodium hydroxide. Dimethylformamide and acetonitrile are added to the aqueous layer and stirred.

The reaction mixture pH is adjusted to 10 to 12 using aqueous sodium hydroxide for precipitating the imatinib base and stirred at 25-35° C. In one embodiment, 10% aqueous sodium hydroxide is used for adjusting the pH.

Step g) involves isolating imatinib base of formula IV.

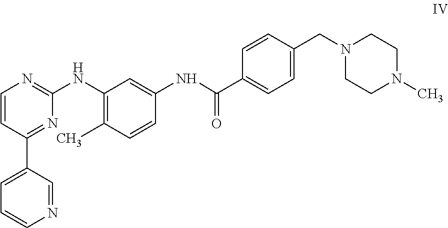 [SMILES: Cc1ccc(NC(=O)c2ccc(CN3CCN(C)CC3)cc2)cc1Nc1nccc(-c2cccnc2)n1]

Imatinib base obtained in Step f) may be isolated by techniques known in art which include, but are not limited to filtration by gravity or by suction, distillation, centrifugation, or slow evaporation and the like. Optionally, the product may be slurry washed with water. The product obtained may be further purified from crystallization dimethylformamide and acetonitrile to provide imatinib base having a purity of greater than about 99.9%.

Step h) involves optionally converting imatinib base to its mesylate salt. Imatinib base obtained may be converted to imatinib mesylate by the processes known in the art or as per the processes disclosed in the present application.

N-(5-amino-2-methylphenyl)-4-(3-pyridyl)-pyrimidine-amine of formula II appears to be one of the mutagenic intermediates/impurities, the presence of which in the end product including imatinib base or its salt may be a threat, if the end product does not provide a negative Ames test (a specific test for mutagenicity; performed according to the OECD Guideline for Testing of Chemicals, 471: Bacterial Reverse Mutation Test, Adopted Jul. 21, 1997). The process of the present disclosure provides a product, which adequately compiles with the regulatory requirements by successfully removing the said mutagenic intermediate compound of formula II.

An embodiment of the present application also provides a process which involves reacting the compound of Formula II with the compound of Formula III

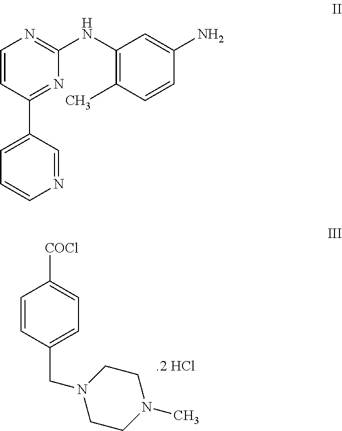 [SMILES: Cc1ccc(N)cc1Nc1nccc(-c2cccnc2)n1 ; CN1CCN(Cc2ccc(C(=O)Cl)cc2)CC1]

in the presence of N-methylmorpholine base and dichloromethane solvent.

In an embodiment, the present application provides a process for preparing imatinib mesylate comprising:

a) reacting N-(5-amino-2-methylphenyl)-4-(3-pyridyl)-pyrimidine-amine of formula II:

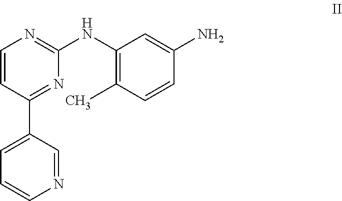 [SMILES: Cc1ccc(N)cc1Nc1nccc(-c2cccnc2)n1]

with 4-((4-methylpiperazin-1-yl)methyl)benzoyl chloride dihydrochloride of the formula III:

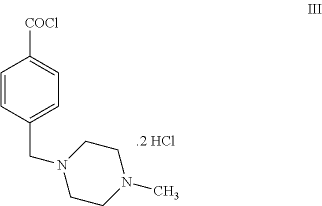 [SMILES: CN1CCN(Cc2ccc(C(=O)Cl)cc2)CC1]

in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof to obtain imatinib base of formula IV; and

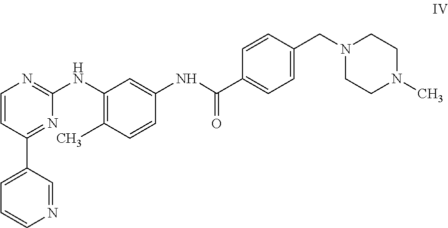 [SMILES: Cc1ccc(NC(=O)c2ccc(CN3CCN(C)CC3)cc2)cc1Nc1nccc(-c2cccnc2)n1]

b) converting the compound of formula IV to its mesylate salt.

In an embodiment, the present application provides a process for preparing substantially pure imatinib base comprising the steps of:

a) providing a solution or suspension of imatinib base in dimethylformamide;

b) adding acetonitrile;

c) maintaining the solution at a temperature of about 30° C. to about 60° C.;

d) optionally cooling the reaction mass; and

e) isolating imatinib base.

The individual steps of the process are detailed herein below. Step a) involves providing a solution or suspension of imatinib base in dimethylformamide. The imatinib base used in the process of the present disclosure may be obtained from processes disclosed in the art or as per the process described in the present application. The solution or suspension of imatinib base in dimethylformamide may be provided by combining imatinib base with dimethylformamide and optionally heating to a temperature of about 40 to about 80° C. Alternately, the solution or suspension of imatinib base in dimethylformamide may be obtained directly from a reaction in which imatinib base is formed. In an embodiment, a solution of imatinib base in dimethylformamide is provided by combining imatinib base with dimethylformamide and heating to a temperature of about 60 to about 65° C. The solution obtained may be optionally filtered and cooled.

Step b) involves adding acetonitrile. Adding acetonitrile to the solution or suspension of imatinib base obtained in Step a) may be carried out at a temperature as maintained in step a), or may be added at a reduced temperature to which the solution or suspension is precooled. In one aspect, acetonitrile is added to the solution of suspension of step a) at a temperature of about 30° C. to about 40° C.

Step c) involves maintaining the solution at a temperature of about 30° C. to about 60° C. The solution of imatinib base obtained in step b) is maintained at a temperature of about 30° C. to about 60° C. In one aspect, the solution is maintained at a temperature of about of 40° C. to about 45° C. The solution may be maintained at the desired temperature for a desired period of time or till the solid isolation is observed. In an embodiment, the reaction mass is maintained at a temperature of about 40° C. to about 45° C. for a time period of about 60 to 90 minutes. The reaction mass of step c) may be directly taken forward for the isolation step e) or may be subjected to step d).

Step d) involves optionally cooling the reaction mass. The reaction mass of step c) may be optionally cooled to a temperature less than 30° to increase the crystallization of the imatinib base.

e) isolating imatinib base. Isolation of the imatinib may be done by techniques known in art which include, but are not limited to, filtration by gravity or by suction, distillation, centrifugation, or slow evaporation, or the like. The product obtained may be dried at a temperature of about 45° C. to about 65° C. to provide imatinib base having a purity of greater than about 99.9%.

The above processes of the present application provides substantially pure imatinib base and imatinib mesylate having purity greater than or equal to about 99.8%, or greater than or equal to about 99.9% by weight as determined using HPLC. Further, the above processes of the present application also provides substantially pure imatinib base and imatinib mesylate containing less than about 0.2% or less than about 0.1% of one or more of the compounds of formula 11, Impurity A, or Impurity B, by weight as determined using HPLC. The present disclosure also provides imatinib base containing less than about 0.002% (20 ppm) of the compound of the formula II. In an embodiment, the present disclosure provides imatinib base containing less than about 0.001% (10 ppm) of the compound of the formula II. Further, the present disclosure provides imatinib mesylate containing less than about 0.001% (10 ppm) of the compound of the formula II.

In an embodiment, the present application provides a process for the preparing alpha-crystalline form of imatinib mesylate, comprising:

a) providing a solution or suspension of imatinib base in a solvent chosen from acetone, methyl isobutyl ketone, or methyl ethyl ketone;

b) heating to a temperature up to the boiling point of the solvent used in the step a);

c) adding methanol;

d) adding methanesulfonic acid;

e) allowing the reaction mass to cool; and

f) isolating the alpha-crystalline form of imatinib mesylate.

The individual steps of the process are detailed below. Step a) involves providing a solution or suspension of imatinib base in a solvent chosen from acetone, methyl isobutyl ketone, or methyl ethyl ketone. The process of providing a solution or suspension of imatinib base in acetone, methyl isobutyl ketone, or methyl ethyl ketone involves combining imatinib base with the solvent chosen or may also be obtained directly from a reaction in which imatinib base is formed. The quantity of solvent used depends on the solvent and the temperature adopted.

Step b) involves heating to a temperature up to the boiling point of the solvent used in step a). The solution or suspension obtained in Step a) is heated to a temperature up to the boiling point of the solvent used. In an embodiment, the suspension obtained in step a) is heated to a temperature of about 55° C. to about 60° C. when acetone is used as the solvent; to a temperature of about 70° C. to about 80° C. if methyl ethyl ketone is used as the solvent, and to a temperature of about 80° C. to about 85° C. if methyl isobutyl ketone is used as the solvent.

Step c) involves adding methanol. Methanol may be added to the reaction mass obtained in step b) to provide a solution. In an embodiment, methanol may be added at the temperature maintained in step b), for providing the solution of imatinib base.

Step d) involves adding methanesulfonic acid. According to the process of the present disclosure, the molar quantity of methanesulfonic acid used may be in the range of about 1.0 to about 1.02 moles, per mole of imatinib base. In embodiments, the molar quantity of methanesulfonic acid used is about 1 mole per mole of imatinib base. In accordance with the present disclosure, methanesulfonic acid can be added either directly or as a solution with a solvent used in step a). In one embodiment, methanesulfonic acid is added as a solution with a solvent used in step a). According to the process of the present disclosure, seeding with crystalline alpha form imatinib mesylate is carried out only after the addition of methanesulfonic acid. The alpha-crystalline form obtained from a process disclosed in the art or from the process of the present disclosure may be used as a seed material and may be used before or after the addition of methanesulfonic acid. The quantity of the seed crystals used herein may be in the range of about 1 to about 8% w/w of alpha-form seeding material, based on the weight of suspended imatinib free base.

Step e) involves allowing the reaction mass to cool. The temperature to which the reaction mass is cooled may range from about 20° C. to about 40° C. to increase the crystallization of the alpha-crystalline form of imatinib mesylate. In embodiments, the cooling may be carried out to temperatures in the range of about 25° C. to about 30° C.

Step f) involves isolating the alpha-crystalline form of imatinib mesylate. Isolation of the alpha-crystalline form obtained in step e) can be done by techniques known in art which include, but are not limited to filtration by gravity or by suction, distillation, centrifugation, or slow evaporation and the like. In embodiments, the obtained alpha-form of the present disclosure is isolated by filtration. The wet product obtained may be dried at temperatures about 55° C. to about 70° C., in one aspect at about 40° C. to about 50° C., with or without vacuum. Drying may be suitably carried out using equipment such as a tray dryer, vacuum oven, air oven, fluidized bed drier, spin flash dryer, flash dryer, or the like.

In an embodiment, the present application provides a process for the preparing alpha-crystalline form of imatinib mesylate, comprising:

a) providing a solution of imatinib base in a halogenated hydrocarbon solvent;

b) adding methanesulfonic acid;

c) maintaining the reaction mass at reflux temperature;

d) allowing the reaction mass to cool; and

e) isolating the alpha-crystalline form of imatinib mesylate.

The individual steps of the process are detailed herein below. Step a) involves providing a solution of imatinib base in a halogenated hydrocarbon solvent. The halogenated hydrocarbon solvents that may be used for providing the solution of imatinib base includes, but are not limited to dichloromethane, chloroform, or the like. The solution of imatinib base may be provided by combining imatinib base with the solvent chosen and heating to temperatures up to the boiling point of the solvent used. The solution of imatinib base may also be obtained directly from a reaction in which imatinib base is formed. In an embodiment, the solution of imatinib base may be provided by combining imatinib base with dichloromethane and heating to a temperature of about 35° C. to about 40° C.

Step b) involves adding methanesulfonic acid. According to the process of the present disclosure, the molar quantity of methanesulfonic acid used may be in the range of about 1.0 to about 1.02 moles, per mole of imatinib base. In embodiments, the molar quantity of methanesulfonic acid used is about 1 mole per mole of imatinib base. In accordance with the present disclosure, methanesulfonic acid can be added either directly or as a solution with an ester solvent to the mixture of step a). Ester solvents that may be used for providing the solution of imatinib base includes, but are not limited to ethyl acetate, butyl acetate, or the like. In one embodiment, methanesulfonic acid can be added in the form of solution in ethyl acetate. The quantity of ethyl acetate used to prepare a solution of methanesulfonic acid may range between about 1 volume to about 5 volumes per gram of imatinib base. The temperature at which methanesulfonic acid can be added to the reaction mass of step b) may range from about 25° C. to a temperature up to the boiling point of the solvent used in step a) and in one aspect may range from about 25° C. to about 40° C. According to the process of the present disclosure, seeding with imatinib mesylate is optional. However, if carried out, the alpha-crystalline form obtained from a process disclosed in the art or from the process of the present disclosure may be used as a seed material and may be used before or after the addition of methanesulfonic acid.

Step c) involves maintaining the reaction mass at reflux temperature. The reaction mass obtained in Step b) may be maintained at the reflux temperature for a desired period of time or till solid separation is observed. In one aspect, the reaction mass is maintained for a time period of about 1 hour to about 5 hours.

Step d) involves allowing the reaction mass to cool. The temperature to which the reaction mass is cooled may range from about 20° C. to about 30° C. to increase the crystallization of the alpha-crystalline form of imatinib mesylate.

Step e) involves isolating the alpha-crystalline form of imatinib mesylate.

Isolation of the alpha-crystalline form obtained in step d) can be done by techniques known in art which include, but are not limited to filtration by gravity or by suction, distillation, centrifugation, or slow evaporation and the like. In embodiments, the obtained alpha-form of the present disclosure is isolated by filtration. The wet product obtained may be dried at a temperature of about 45° C. to provide alpha-crystalline form of imatinib mesylate.

In an embodiment, the present application provides a process for the preparing alpha-crystalline form of imatinib mesylate, comprising:

a) providing a suspension of imatinib base in a solvent mixture chosen from methyl ethyl ketone and acetone or methyl isobutyl ketone and acetone;

b) heating the suspension to a temperature up to the boiling point of the solvent used in the step a);

c) adding methanesulfonic acid at the same temperature;

d) allowing the reaction mass to cool; and

e) isolating the alpha-crystalline form of imatinib mesylate.

The individual steps of the process are detailed below. Step a) involves providing a suspension of imatinib base in a solvent mixture chosen from methyl ethyl ketone and acetone or methyl isobutyl ketone and acetone. The suspension of imatinib base may be provided by combining imatinib base with a solvent system chosen from methyl ethyl ketone and acetone or methyl isobutyl ketone and acetone.

Step b) involves heating the suspension to a temperature up to the boiling point of the solvent system in step a). The reaction mass obtained in Step a) is heated to a temperature up to the boiling point of the solvent system in step a). In an embodiment, the reaction mass obtained in step a) is heated to a temperature up to about 60° C. to about 65° C.

Step c) involves adding methanesulfonic acid at the same temperature. According to the process of the present disclosure, the molar quantity of methanesulfonic acid used may be in the range of about 1.0 to about 1.02 moles, per mole of imatinib base. In embodiments, the molar quantity of methanesulfonic acid used is about 1 mole per mole of imatinib base. In accordance with the present disclosure, methanesulfonic acid can be added either directly or as a solution with solvent used in step a). In an embodiment a solution of methanesulfonic acid is added as a solution in methyl isobutyl ketone or methyl ethyl ketone at a temperature of about 60° C. to about 65° C. and over a time period of about 60 minutes to about 90 minutes and maintained at the same temperature for about 15 minutes to about 30 minutes. According to the process of the present disclosure, the seeding with alpha-crystalline form imatinib mesylate is carried out prior to the addition of the methanesulfonic acid solution. The alpha-crystalline form obtained from a process disclosed in the art or from the process of the present disclosure may be used as a seed material.

Step d) involves allowing the reaction mass to cool. The temperature to which the reaction mass is cooled may range from about 20° C. to about 35° C. to increase the crystallization of the alpha-crystalline form of imatinib mesylate. In embodiments, the cooling may be carried out to temperatures in the range of about 25° C. to about 30° C.

Step e) isolating the alpha-crystalline form imatinib mesylate. Isolation of the alpha-crystalline form obtained in step d) can be done by techniques known in art which include, but are not limited to filtration by gravity or by suction, distillation, centrifugation, or slow evaporation and the like. In embodiments, the obtained alpha-form of the present disclosure is isolated by filtration. The wet product obtained may be dried at a temperature less than 55° C. to provide alpha-crystalline form of imatinib mesylate.

The use of acetone alone as the solvent for the preparation of alpha-crystalline form of imatinib mesylate resulted in the product with the content of residual solvent acetone greater than the ICH limit and when prepared by the process of the disclosure which involves the use of acetone along with methyl isobutyl ketone or methyl ethyl ketone results in alpha-crystalline form of imatinib mesylate with the content of acetone as per the ICH limit.

The above processes for the preparation of alpha-crystalline form of imatinib mesylate provides substantially pure imatinib base and imatinib mesylate having purity greater than or equal to about 99.8%, or greater than or equal to about 99.9%, by weight as determined using HPLC. Further, the present application also provides alpha-crystalline form of imatinib mesylate containing less than about 0.001% of the compound of the formula II. The impurities in imatinib base or imatinib mesylate may be analyzed using various methods. Representative useful HPLC methods are described below.

Further, the applicants have observed that cyanamide on long standing dimerizes to give 2-cyanoguanidine (dicyandiamide), which could participate in the subsequent reactions for the preparation of imatinib and results in the formation of impurity C, which gets converted to corresponding guanidine impurity of imatinib i.e., impurity D. In an embodiment, the processes for the preparation of imatinib base and imatinib mesylate as described in the present application are capable of controlling the impurity C and impurity D to contents less than about 0.15% by HPLC. In an embodiment, the present application provides imatinib mesylate containing less than about 0.15% of the compound of the impurity C and less than about 0.15% of impurity D.

Further, the applicants have observed during the synthesis of 4-((4-methylpiperazin-1-yl)methyl)benzoyl chloride, an intermediate for the preparation of Imatinib, potential impurities viz., impurity E, impurity F and impurity G were obtained, which could participate in the subsequent reactions for the preparation of imatinib and results in the formation of corresponding impurities viz., impurity H, impurity I, and impurity J. In an embodiment, the processes for the preparation of imatinib base and imatinib mesylate as described in the present application are capable of controlling the impurity H, impurity I, and impurity J to contents less than about 0.15% by HPLC. In an embodiment, the present application provides imatinib mesylate containing less than about 0.15% or less than about 0.1% of the compound of the impurity H, less than about 0.15% of the compound of the impurity I, and less than about 0.15% or less than about 0.1% of impurity J.

In an embodiment, the present application provides substantially pure imatinib base and imatinib mesylate containing less than about 0.2% or less than about 0.1% of one or more of the compounds of formula II, Impurity A, Impurity B, impurity C, impurity D, impurity H, impurity I, or impurity J by weight as determined using HPLC. The impurities A, B, C, D, H, I and J may be analyzed by a method as described below as “Method 1”.

TABLE 1

| Impurity | Structure |
|---|---|
| Impurity A | 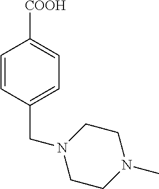 [SMILES: CN1CCN(Cc2ccc(C(=O)O)cc2)CC1] |
| Impurity B | 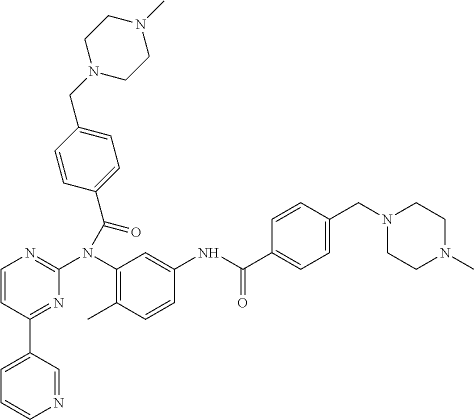 [SMILES: Cc1ccc(NC(=O)c2ccc(CN3CCN(C)CC3)cc2)cc1N(C(=O)c1ccc(CN2CCN(C)CC2)cc1)c1nccc(-c2cccnc2)n1] |
| Impurity C | 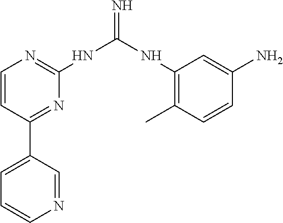 [SMILES: Cc1ccc(N)cc1NC(=N)Nc1nccc(-c2cccnc2)n1] |
| Impurity D | 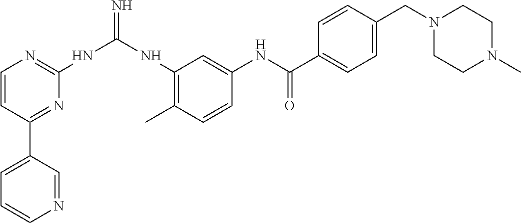 [SMILES: Cc1ccc(NC(=O)c2ccc(CN3CCN(C)CC3)cc2)cc1NC(=N)Nc1nccc(-c2cccnc2)n1] |
| Impurity E | 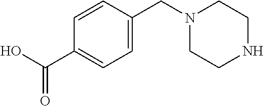 [SMILES: O=C(O)c1ccc(CN2CCNCC2)cc1] |
| Impurity F | 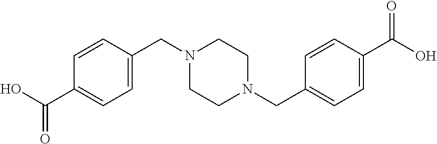 [SMILES: O=C(O)c1ccc(CN2CCN(Cc3ccc(C(=O)O)cc3)CC2)cc1] |
| Impurity G | 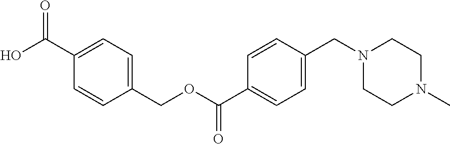 [SMILES: CN1CCN(Cc2ccc(C(=O)OCc3ccc(C(=O)O)cc3)cc2)CC1] |
| Impurity H | 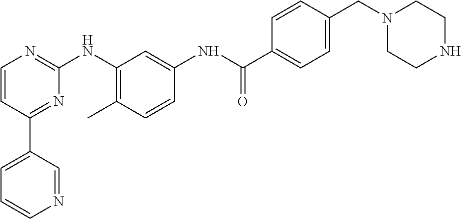 [SMILES: Cc1ccc(NC(=O)c2ccc(CN3CCNCC3)cc2)cc1Nc1nccc(-c2cccnc2)n1] |
| Impurity I | 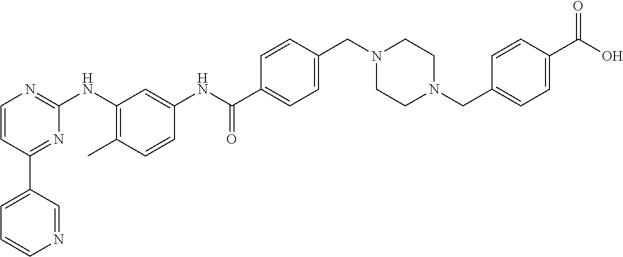 [SMILES: Cc1ccc(NC(=O)c2ccc(CN3CCN(Cc4ccc(C(=O)O)cc4)CC3)cc2)cc1Nc1nccc(-c2cccnc2)n1] |
| Impurity J | 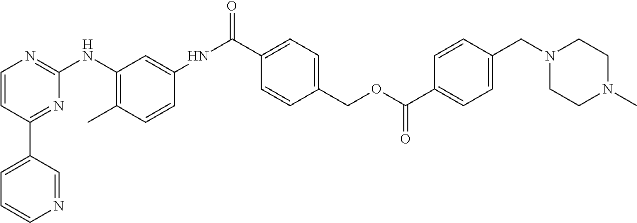 [SMILES: Cc1ccc(NC(=O)c2ccc(COC(=O)c3ccc(CN4CCN(C)CC4)cc3)cc2)cc1Nc1nccc(-c2cccnc2)n1] |

Method 1. Imatinib base or imatinib mesylate may be analyzed by HPLC utilizing the following conditions:
Column: Waters Symmetry® C18, (150×4.6) mm, 5 μm
Column temperature: 35±2° C.
Injection volume: 10 μL
Elution: Gradient
Run time: 65 minutes
Buffer: Dissolved 7.5 g of 1-octanesulfonic acid sodium salt anhydrous in 1000 mL of water. Adjusted the pH to 2.3±0.05 with phosphoric acid solution. Filtered the solution using 0.45 micron filter and degassed.
Mobile Phase A: Buffer
Mobile Phase B: Mixture of methanol: acetonitrile in the volume ratio of 75:25 and degassed
Flow rate: 1.0 mL/min.
Wavelength of detection: 234 nm UV.
p Diluent: methanol: water (55:45) v/v
Gradient program:

| Minutes | % Mobile Phase A | % Mobile Phase B |
|---|---|---|
| 0 | 50 | 50 |
| 20 | 50 | 50 |
| 25 | 45 | 55 |
| 35 | 45 | 55 |
| 45 | 30 | 70 |
| 56 | 30 | 70 |
| 57 | 50 | 50 |
| 65 | 50 | 50 |

Using this method, the relative retention times (where the compound of Formula I=1) listed in Table 2 are obtained for certain process-related impurities.

TABLE 2

| Impurity | RRT |
|---|---|
| 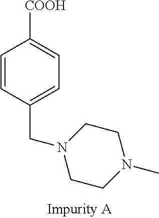 [SMILES: CN1CCN(Cc2ccc(C(=O)O)cc2)CC1] | 0.17 |
| 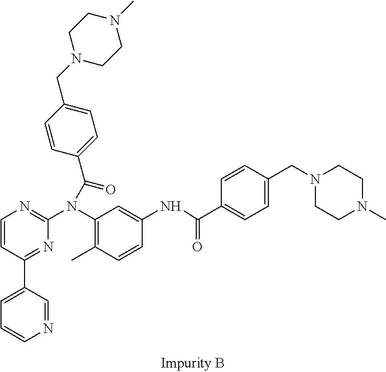 [SMILES: Cc1ccc(NC(=O)c2ccc(CN3CCN(C)CC3)cc2)cc1N(C(=O)c1ccc(CN2CCN(C)CC2)cc1)c1nccc(-c2cccnc2)n1] | 1.24 |

Method 2. Imatinib base or imatinib mesylate may also be analyzed by HPLC utilizing the following conditions:
Column: Waters Symmetry® C18, (150×4.6) mm, 5 μm
Column temperature: 35±2° C.
Injection volume: 20 μL
Elution: Gradient
Run time: 35 minutes
Buffer: Dissolved 7.5 g of 1-octanesulfonic acid sodium salt anhydrous and 1.36 g of potassium dihydrogen orthophosphate in 1000 mL of water. Adjusted the pH to 2.3±0.05 with phosphoric acid solution. Filtered the solution using 0.45 micron filter and degassed.
Mobile Phase A: Buffer
Mobile Phase B: Mixture of methanol: acetonitrile in the volume ratio of 75:25 and degassed
Flow rate: 1.0 mL/min.
Wavelength of detection: 267 nm UV.
Diluent: methanol: water (55:45) v/v
Gradient program:

| Minutes | % Mobile Phase A | % Mobile Phase B |
|---|---|---|
| 0 | 47 | 53 |
| 10 | 47 | 53 |
| 15 | 45 | 55 |
| 20 | 40 | 60 |
| 23 | 30 | 70 |
| 28 | 20 | 80 |
| 29 | 47 | 53 |
| 35 | 47 | 53 |

Using this method, the relative retention times (where the compound of Formula I=1) listed in Table 3 are obtained for certain process-related impurities.

TABLE 3

| Impurity | RRT |
|---|---|
| Formula II | 0.34 |

### DEFINITIONS

The following definitions are used in connection with the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like.

An “aliphatic alcohol solvent” is an organic solvent containing an aliphatic carbon bound to a hydroxyl group. “C₁-C₆ Aliphatic alcohol solvents” include but are not limited to methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, glycerol, and the like.

An “ester solvent” is an organic solvent containing a carboxyl group —(C═O)—O— bonded to two other carbon atoms. “Ester solvents” include but are not limited to ethyl acetate, n-propyl acetate, n-butyl acetate, isobutyl acetate, t-butyl acetate, and the like.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, chloroform, carbon tetrachloride, and the like. Polymorphs are different solids sharing the same molecular formula, yet having distinct physical properties when compared to other polymorphs of the same formula.

A polymorphic form may be described by reference to patterns, spectra, or other graphical data as “substantially” shown or depicted in a figure, or by one or more data points. It will be appreciated that patterns, spectra, and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation, the equipment used, the sample preparation protocol, preferred packing and orientations, the radiation source, operator error, method and length of data collection, and the like. However, those of ordinary skill in the art will be able to compare the figures herein with patterns, etc. generated for an unknown form of, in this case, imatinib mesylate, and confirms its identity. The same holds true for other techniques which may be reported herein.

In addition, where a reference is made to a figure, it is permissible to, and this document includes and contemplates, the selection of any number of data points illustrated in the figure that uniquely define that crystalline form, within any associated and recited margin of error, for purposes of identification.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present invention. While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EXAMPLES

### Example 1

Preparation of 4-[(4-methyl-1-piperazinyl)methyl]-N-[4-methyl-3-[(4-(3-pyridinyl)-2-pyrimidinyl]amino]-phenyl]benzamide (imatinib base). Charged dichloromethane (450 mL) into a round bottom flask and cooled to −5 to 0° C. N-(5-amino-2-methyl phenyl)-4-(3-pyridinyl)2-pyrimidine-amine (30 g) and N-methyl morpholine (83.1 mL) are charged to the flask and stirred. Charged dichloromethane (300 mL) into a second round bottom flask, cooled to −5 to 0° C. and added 4-((4-methylpiperazin-1-yl)methyl)benzoyl chloride dihydrochloride (45.8 g). Charged the solution in the first round bottom flask to the second flask slowly and stirred at a temperature of 0-5° C. for about 60 minutes. Charged 10% sodium hydroxide solution (310 mL) to the reaction mass (pH of the reaction mass is ˜11.2). Stirred the reaction mass at 24° C. for 10 minutes and separated the layers. Extracted the reaction mass with dichloromethane (300 mL) and combined the dichloromethane layers. Charged water (1200 mL) to the combined layers and adjusted the pH to 4.5-50 using 20% acetic acid solution. Separated the layers and washed the aqueous layer with dichloromethane (3×150 mL). Charged dimethylformamide (60 mL) and acetonitrile (450 mL) to the aqueous layer, adjust the pH of the reaction mass to above 10 using 10% sodium hydroxide solution, and stirred the reaction mass for 1 hour. The solid was collected by filtration, washed with water (90 mL), followed by acetonitrile (90 mL), and dried by suction on the filter. The product obtained is charged in a round bottom flask, added water (300 mL) and stirred for 30 minutes. Collected the product by filtration, washed with water (300 mL), followed by acetonitrile (90 mL), dried by suction on the filter, and dried in the oven at 55° C. under high vacuum. Yield: 35.5 g (66.8%).

### Example 2

Purification of 4-[(4-methyl-1-piperazinyl) methyl]-N-[4-methyl-3-[[4-(3-pyridinyl)-2-pyrimidinyl]amino]-phenyl]benzamide (imatinib base) using dimethylformamide and acetonitrile. Charged imatinib base (35.0 g) and dimethylformamide (175 mL) into a round bottom flask and heated the reaction mixture to 60-65° C. to obtain a clear solution. Filtered the reaction mass and charged the filtrate into a round bottom flask containing acetonitrile (700 mL). Heated the reaction mass to 40-45° C. and maintained for 1 hour at the same temperature. The product obtained was collected by filtration at the same temperature, washed with acetonitrile (70 mL), dried by suction on the filter, and dried in oven at 48° C. under high vacuum. Yield: 31.8 g (91.0%) Purity: 99.95%, Formula II: 0.0008%, Impurity A: Not detected, Impurity B: Not detected.

### Example 3

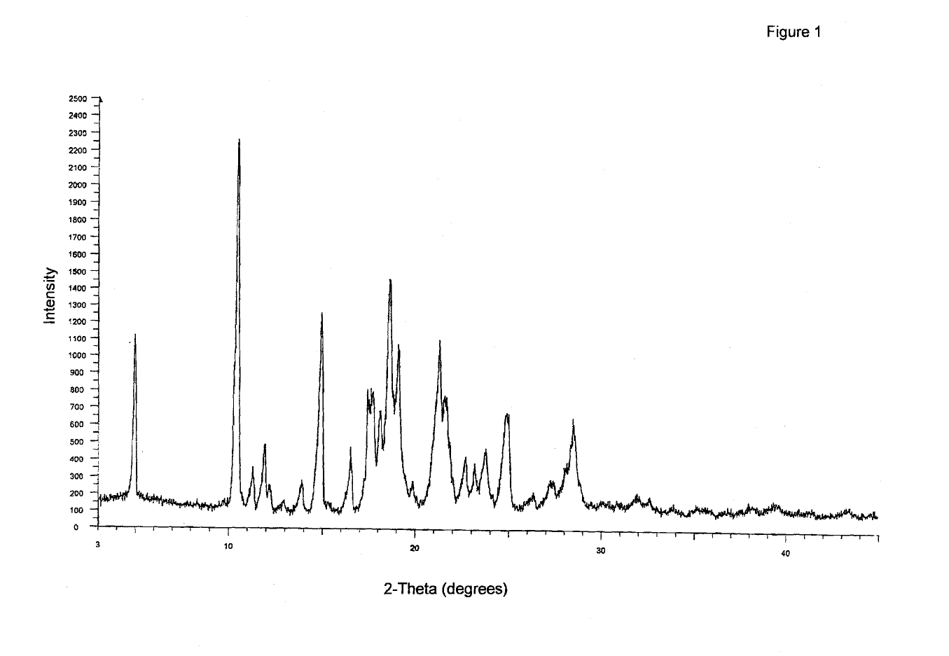

Preparation of alpha-crystalline form of imatinib mesylate using acetone and methyl isobutyl ketone. Methyl isobutyl ketone (800 mL), acetone (400 mL) and imatinib base (40 g) are charged in to the round bottom flask. The reaction mixture is heated to 60-65° C., seeded with the alpha-crystalline form of imatinib mesylate (2 g) and stirred for 15 minutes. Methanesulfonic acid solution (5.26 mL of methanesulfonic acid in methyl isobutyl ketone (400 mL)) is added to the reaction mixture slowly over a period of 60 minutes at the same temperature. Stirred the reaction mixture for 20 minutes and slowly allowed the reaction mixture to cool to a temperature of 25-30° C. Stirred the reaction mixture for 60 minutes, collected the product by filtration under nitrogen atmosphere, washed with methyl isobutyl ketone (400 mL), dried by suction on the filter for 10-15 minutes, and dried in the oven at 48-50° C. under high vacuum. Yield: 46.0 g (96.24%), Purity: 99.88%, Formula II: 0.0012%, Impurity A: not detected, Impurity B: not detected, XRPD pattern: FIG. 1, DSC: 227.0° C., TGA: 0.1129%, acetone: 1714 ppm, methyl isobutyl ketone: 988 ppm.

### Example 4

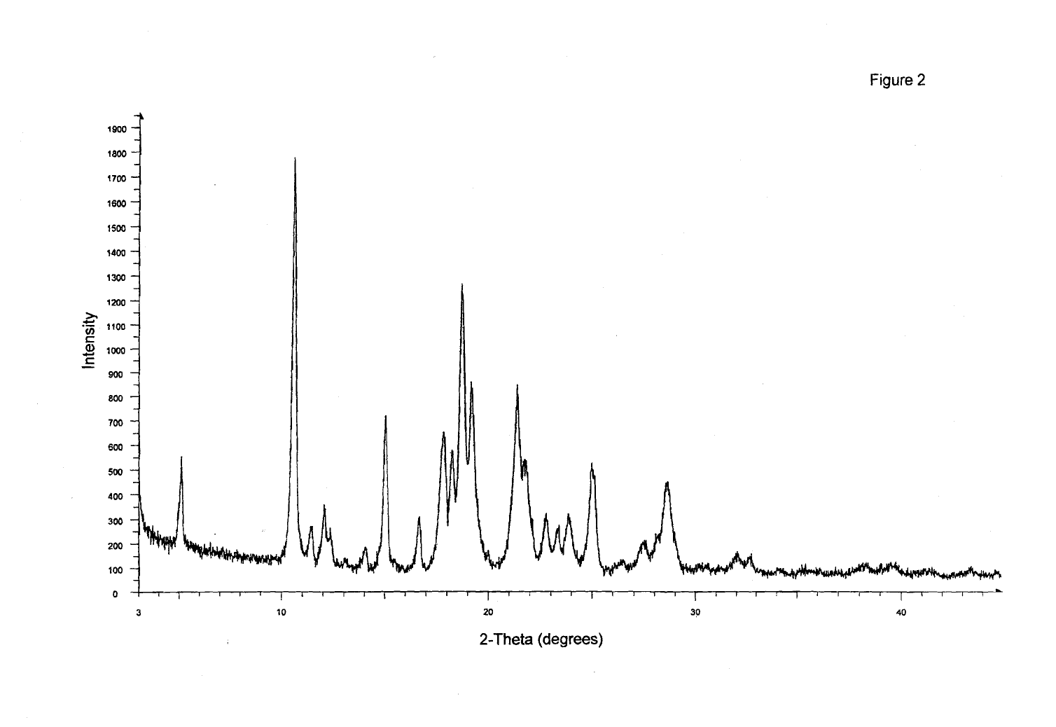

Preparation of alpha-crystalline form of imatinib mesylate using dichloromethane and ethyl acetate. Dichloromethane (105 mL) and imatinib base (3 g) are charged into a round bottom flask and heated to reflux temperature. Dichloromethane (45 mL) is added to the reaction mixture and maintained at reflux temperature of 37° C. to get a clear solution. Methanesulfonic acid solution (obtained from methanesulfonic acid (0.39 mL) in ethyl acetate (6 mL)) is added to the reaction mixture slowly over a period of 15 minutes and maintained at reflux temperature for one hour to precipitate the solid. The temperature of the reaction mixture is gradually decreased to about 25-30° C. and stirred for about 1 hour. The product obtained is Collected the product by filtration under nitrogen atmosphere, washed with dichloromethane (30 mL), dried by suction on the filter for about 10 minutes and dried in vacuum oven at the temperature about 40-45° C. under high vacuum for about 3-4 hours. Yield: 2.4 g (67%), XRPD pattern: FIG. 2.

### Example 5

Preparation of alpha-crystalline form of imatinib mesylate using acetone and methanol. Acetone (150 mL) and imatinib base (3 g) are charged into the round bottom flask and heated to about 50-55° C. to obtain a solution. Methanol (10mL) is added to the reaction mixture, seeded with the alpha-crystalline form of imatinib mesylate (150 mg), and stirred. Methanesulfonic acid solution (obtained from methanesulfonic acid (0.39 mL) in acetone (10 mL)) is added to the reaction mixture slowly for 15 minutes at the temperature about 55-60° C. The temperature of the reaction mixture is gradually decreased to about 25-30° C. and stirred for about 1 hour. The product obtained is collected by filtration, washed with acetone (10 mL), dried by suction on the filter for about 10 minutes, and dried in vacuum oven at the temperature of 50° C. under vacuum. Yield: 3.1 g (87.6%).

### Example 6

Preparation of alpha-crystalline form of imatinib mesylate using methyl ethyl ketone (MEK) and methanol. Imatinib base (3 g) and methyl ethyl ketone (90 mL) are charged into a round bottom flask and heated to 72° C. Methanol (17.5 mL) is added to the reaction mixture and stirred to obtain a clear solution. Methanesulfonic acid solution (obtained from methanesulfonic acid (0.39 mL) in methyl ethyl ketone (10 mL)) is added to the reaction mixture slowly for 20 minutes at the same temperature and seeded with the alpha-crystalline form of imatinib mesylate (150 mg). The reaction mixture is allowed to cool to a temperature of 25-30° C. and stirred for about 90 minutes. The product obtained is collected by filtration, washed with methyl ethyl ketone (10 mL), dried by suction on the filter for about 10 minutes, and dried in vacuum oven at the temperature of 50° C. under vacuum. Yield: 2.9 g (81.9%).

### Example 7

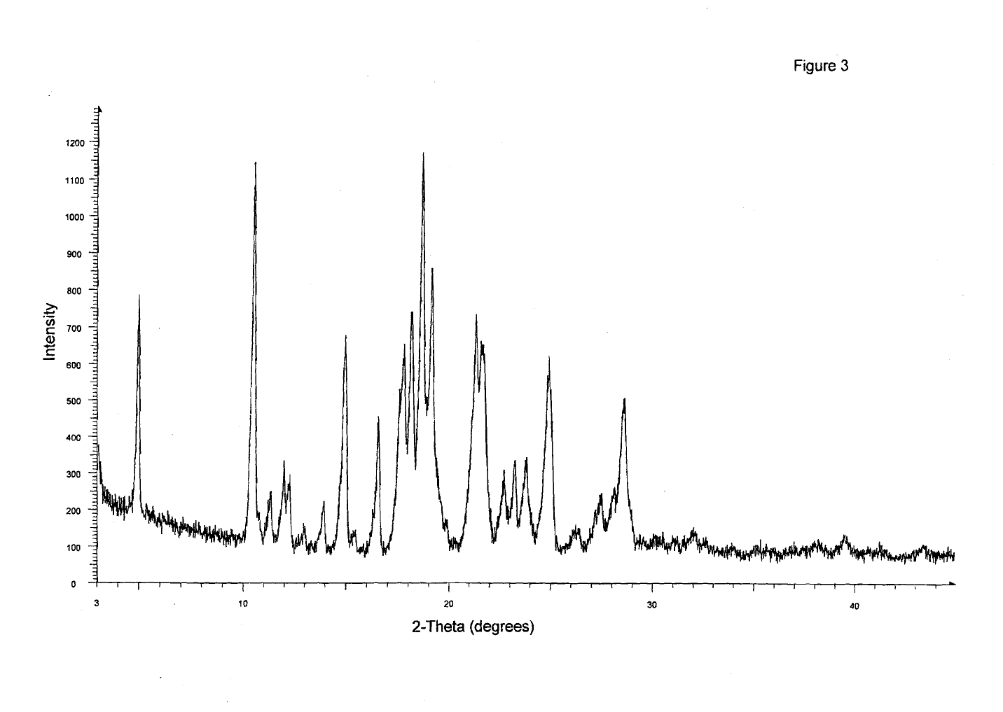

Preparation of alpha-crystalline form of imatinib mesylate using methyl isobutyl ketone (MIBK) and methanol. Imatinib base (3 g) and methyl isobutyl ketone (150 mL) are charged into a round bottom flask and heated to 80-85° C. Methanol (22.5 mL) is added to the reaction mixture and stirred to obtain a clear solution. Methanesulfonic acid solution (obtained from methanesulfonic acid (0.39 mL) in methyl isobutyl ketone (10 mL)) is added to the reaction mixture slowly for 15 minutes at the same temperature. The reaction mixture is allowed to cool to a temperature of 25-30° C. and stirred for about 30 minutes. The product obtained is collected by filtration, washed with methyl isobutyl ketone (10 mL), dried by suction on the filter for about 10 minutes, and dried in vacuum oven at the temperature of 50° C. under vacuum. Yield: 2.8 g (79%), methyl isobutyl ketone content: 2792 ppm, XRPD pattern: FIG. 3.

### Example 8

Preparation of alpha-crystalline form of imatinib mesylate using acetone. Imatinib base (10 g) and acetone (500 mL) are charged in to the round bottom flask, heated to about 55-60° C., stirred for about 25 minutes. The reaction mixture is seeded with the alpha-crystalline form of imatinib mesylate (150 mg) and added the methanesulfonic acid solution (obtained from methanesulfonic acid (1.3 mL) in acetone (33 mL)) to the reaction mixture slowly for 10 minutes. The reaction mixture is allowed to cool to a temperature of 25-30° C. and stirred for about 1 hour. The product obtained is collected by filtration, washed with acetone (33 mL), dried by suction on the filter, and dried in vacuum oven at the temperature of about 50° C. under vacuum. Yield: 3.0 g (94%), acetone: 5503 ppm.

### Example 9

Preparation of 4-[(4-methyl-1-piperazinyl)methyl]-N-[4-methyl-3-[(4-(3-pyridinyl)-2-pyrimidinyl]amino]-phenyl]benzamide (imatinib base). Dichloromethane (750 mL) is charged into a round bottom flask and cooled to a temperature between −5 to 5° C. N-(5-Amino-2-methyl phenyl)-4-(3-pyridinyl)2-pyrimidine-amine (50 g) is charged to the flask and stirred. Dichloro methane (500 mL) is charged into a second round bottom flask, cooled to a temperature between −5 to 5° C., 4-((4-methylpiperazin-1-yl)methyl)benzoyl chloride dihydrochloride (76.23 g) is added, and stirred. The solution in the first round bottom flask is charged to the second flask slowly, dichloromethane (250 mL) added, and the reaction mixture is stirred. N-methyl morpholine (138.8 mL) is slowly added at a temperature between −5 to 5° C. over a period of 10 minutes and the reaction mixture is maintained below a temperature of 15° C. for about 1 hour. 10% Sodium hydroxide solution (500 mL) is charged to the reaction mass (pH of the reaction mass is ˜12.2), the reaction mass is allowed to warm to a temperature of about 25 to 30° C. The reaction mass Is stirred for 10 minutes and the layers are separated. The aqueous slayer is extracted with dichloromethane (500 mL) and the dichloromethane layers are combined. Water (2000 mL) and 20% acetic acid solution (500 mL) are charged into a round bottom flask and stirred. The combined layers are charged to the acidic aqueous layer and stirred (pH of the reaction mass ˜4.75). The layers are separated and the aqueous layer is washed with dichloromethane (3×250 mL). The aqueous lawyer is charged into a round bottom flask, dimethylformamide (100 mL) and acetonitrile (750 mL) are added, and the pH of the reaction mass is adjusted with stirring to ˜12.35 using 10% sodium hydroxide solution. The reaction mass Is stirred for 1 hour, the product is collected by filtration, washed with water (150 mL) followed by acetonitrile (150 mL), and dried by suction. The product obtained is charged in a round bottom flask, water (1500 mL) is added, and stirred for about 30 minutes. The product is collected by filtration, washed with water (500 mL) followed by acetonitrile (150 mL), dried by suction on the filter, and dried in the oven at 50° C. under high vacuum. Yield: 57 g (64.04%), HPLC Purity: 99.74%

Imatinib base (50.0 g) and dimethylformamide (250 mL) are charged into a round bottom flask and the reaction mixture is heated to 55-60° C. to obtain a clear solution. The reaction mass is filtered, the filtrate is charged into a round bottom flask, and heated to a temperature of about 50° C. Acetonitrile (1000 mL) is charged and the reaction mixture is stirred at a temperature of about 40-45° C. for 1 hour. The product obtained is collected by filtration at the same temperature, washed with acetonitrile (100 mL), dried by suction on the filter, and dried in oven at 50° C. under high vacuum. Yield:45.8 g (91.6%), HPLC Purity: >99.8%, Formula II: 20 ppm, Impurity A: 0.015%, Impurity B: 0.03%, Impurity C: Not detected, Impurity D: Not detected, Impurity H: 60 ppm, Impurity I: 60 ppm and Impurity J: <0.1%.

### Example 10

Preparation of alpha-crystalline form of imatinib mesylate using acetone and methyl isobutyl ketone. Imatinib base (30 g) and acetone (300 mL) are charged in to a round bottom flask. Methyl isobutyl ketone (600 mL) Is added, the reaction mixture is heated to 60-65° C., seeded with the alpha-crystalline form of imatinib mesylate (1.5 g), and stirred for 30 minutes. Methanesulfonic acid solution (6.12 g of methanesulfonic acid in methyl isobutyl ketone (300 mL)) is added to the reaction mixture slowly over a period of 60 minutes at the same temperature. The reaction mixture Is stirred for about 2 hours and the reaction mixture is allowed to cool to a temperature of 25-30° C. The reaction mixture Is stirred for 60 minutes at a temperature of 25-30° C., the product collected by filtration under nitrogen atmosphere, washed with methyl isobutyl ketone (300 mL), dried by suction on the filter, and dried in the oven at 62° C. under high vacuum. Yield: 35.2 g (94%), Purity: 99.89%.

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A process comprising:
a) reacting N-(5-amino-2-methylphenyl)-4-(3-pyridyl)-pyrimidine-amine of formula II: 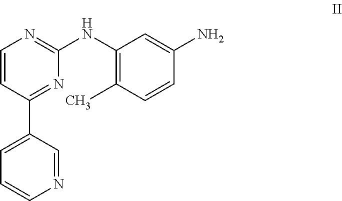 [SMILES: Cc1ccc(N)cc1Nc1nccc(-c2cccnc2)n1]
with 4-((4-methylpiperazin-1-yl)methyl)benzoyl chloride dihydrochloride of the formula III: 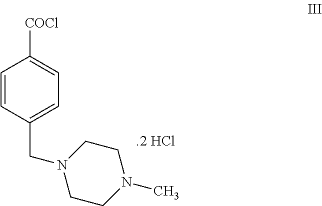 [SMILES: CN1CCN(Cc2ccc(C(=O)Cl)cc2)CC1]
in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof to obtain imatinib base of formula IV; and 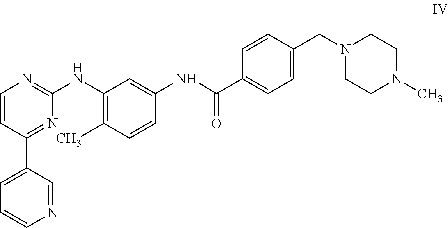 [SMILES: Cc1ccc(NC(=O)c2ccc(CN3CCN(C)CC3)cc2)cc1Nc1nccc(-c2cccnc2)n1]
b) converting the compound of formula IV to its mesylate salt.

2. The process of claim 1, wherein in step a) is carried out at a temperature less than 35° C.

3. A process comprising:
a) reacting N-(5-amino-2-methylphenyl)-4-(3-pyridyl)-pyrimidine-amine of formula II: 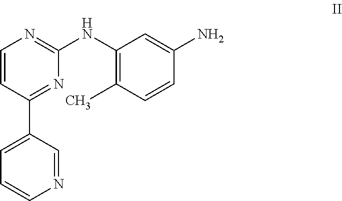 [SMILES: Cc1ccc(N)cc1Nc1nccc(-c2cccnc2)n1]
with 4-((4-methylpiperazin-1-yl)methyl)benzoyl chloride dihydrochloride of the formula III: 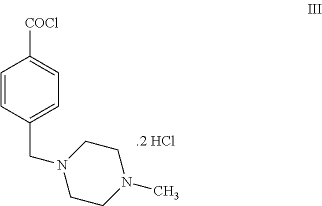 [SMILES: CN1CCN(Cc2ccc(C(=O)Cl)cc2)CC1]
in the presence of a base chosen from N-methylmorpholine, potassium phosphate, or sodium hydrogen phosphate and an organic solvent chosen from dichloromethane, chloroform, N-methylpyrrolidine, dimethylformamide, or a mixture thereof;
b) adjusting the reaction mass pH to 10-12 using aqueous sodium hydroxide;
c) extracting the reaction mass with dichloromethane;
d) adding water to the dichloromethane layer and adjusting the pH to 4-5 using an acid chosen from acetic acid, hydrochloric acid, oxalic acid, or citric acid;
e) separating the aqueous layer and washing with dichloromethane;
f) adding dimethylformamide and acetonitrile to the aqueous layer and adjusting the pH to 10-12 using aqueous sodium hydroxide;
g) isolating imatinib base of formula IV; and 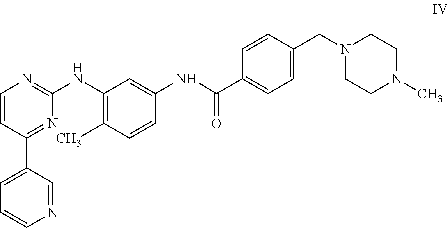 [SMILES: Cc1ccc(NC(=O)c2ccc(CN3CCN(C)CC3)cc2)cc1Nc1nccc(-c2cccnc2)n1]
h) optionally converting the compound of formula IV to its mesylate salt.

4. The process of claim 3, wherein in step a) the amount of base used is from about 1 to about 10 molar equivalents, per molar equivalent of the compound of formula II.

5. The process of claim 4, wherein about 6 to about 8 moles of N-methylmorpholine per molar equivalent of the compound of formula II is used as the base.

6. The process of claim of claim 3, wherein in step a) the amount of compound of formula III is from about 1 to about 1.5 molar equivalents, per molar equivalent of the compound of formula II.

7. A process comprising:
a) providing a solution or suspension of imatinib base in dimethylformamide;
b) adding acetonitrile;
c) maintaining the solution at a temperature of about 30° C. to about 60° C.;
d) optionally cooling the reaction mass; and
e) isolating imatinib base.

8. The process of claim 7, wherein in step a) a solution or suspension of imatinib base in dimethylformamide is used.

9. The process of claim 8, wherein in step c) the solution is maintained at a temperature of about of 40° C. to about 45° C.

10. The process of claim 9, wherein, the reaction mass is maintained at a temperature of about 40° C. to about 45° C. for a time period of about 60 to 90 minutes.

11. A process comprising:
a) providing a solution or suspension of imatinib base in a solvent chosen from acetone, methyl isobutyl ketone, or methyl ethyl ketone;
b) heating to a temperature up to the boiling point of the solvent used in the step a);
c) adding methanol;
d) adding methanesulfonic acid;
e) allowing the reaction mass to cool; and
f) isolating the alpha-crystalline form of imatinib mesylate.

12. The process of claim 11, wherein in step d) the molar quantity of methanesulfonic acid is about 1.0 to about 1.02 moles, per mole of imatinib base.

13. The process of claim 11 further comprising seeding with crystalline alpha form imatinib mesylate before or after the addition of methanesulfonic acid.

14. A process comprising:
a) providing a solution of imatinib base in a halogenated hydrocarbon solvent;
b) adding methanesulfonic acid;
c) maintaining the reaction mass at reflux temperature;
d) allowing the reaction mass to cool; and
e) isolating the alpha-crystalline form of imatinib mesylate.

15. The process of claim 14, wherein in step a) the halogenated hydrocarbon solvent comprises dichloromethane or chloroform.

16. The process of claim 14, wherein in step b) the molar quantity of methanesulfonic acid used is from about 1.0 mole to about 1.02 moles, per mole of imatinib base.

17. The process of claim 14, wherein in step d) temperature to which the reaction mass is cooled is from about 20° C. to about 30° C.

18. A process comprising:
a) providing a suspension of imatinib base in a solvent mixture chosen from methyl ethyl ketone and acetone or methyl isobutyl ketone and acetone;
b) heating the suspension to a temperature up to the boiling point of the solvent used in the step a);
c) adding methanesulfonic acid at the same temperature;
d) allowing the reaction mass to cool; and
e) isolating the alpha-crystalline form of imatinib mesylate.

19. The process of claim 18, wherein in step c) the molar quantity of methanesulfonic acid used is from about 1.0 mole to about 1.02 moles, per mole of imatinib base.

20. The process of claim 18, wherein in step d) the temperature to which the reaction mass is cooled is from about 20° C. to about 35° C.

21. The process of claim 20, wherein the cooling temperatures is from about 25° C. to about 30° C.

22. Imatinib mesylate of claim 1 having a purity of greater than 99.8% and containing less than about 0.1% of one or more of the compounds given in the table below:

| Code | Structure |
|---|---|
| Impurity A | 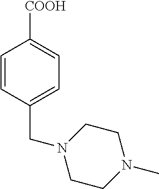 [SMILES: CN1CCN(Cc2ccc(C(=O)O)cc2)CC1] |
| Impurity B | 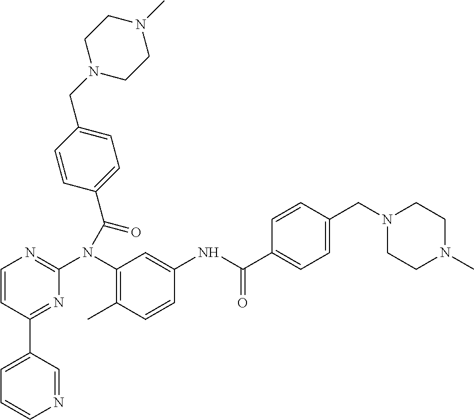 [SMILES: Cc1ccc(NC(=O)c2ccc(CN3CCN(C)CC3)cc2)cc1N(C(=O)c1ccc(CN2CCN(C)CC2)cc1)c1nccc(-c2cccnc2)n1] |
| Impurity C | 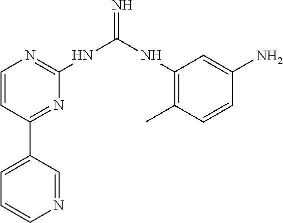 [SMILES: Cc1ccc(N)cc1NC(=N)Nc1nccc(-c2cccnc2)n1] |
| Impurity D | 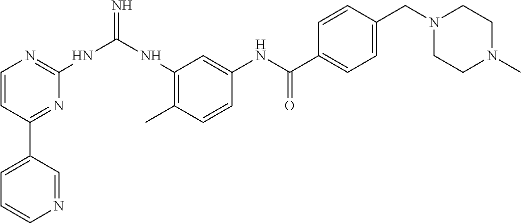 [SMILES: Cc1ccc(NC(=O)c2ccc(CN3CCN(C)CC3)cc2)cc1NC(=N)Nc1nccc(-c2cccnc2)n1] |
| Impurity H | 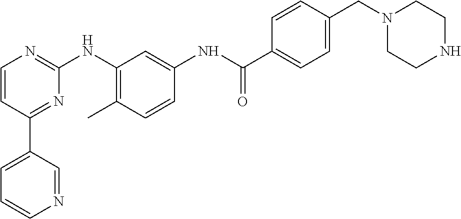 [SMILES: Cc1ccc(NC(=O)c2ccc(CN3CCNCC3)cc2)cc1Nc1nccc(-c2cccnc2)n1] |
| Impurity I | 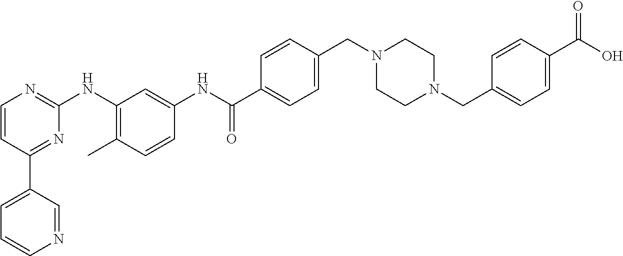 [SMILES: Cc1ccc(NC(=O)c2ccc(CN3CCN(Cc4ccc(C(=O)O)cc4)CC3)cc2)cc1Nc1nccc(-c2cccnc2)n1] |
| Impurity J | 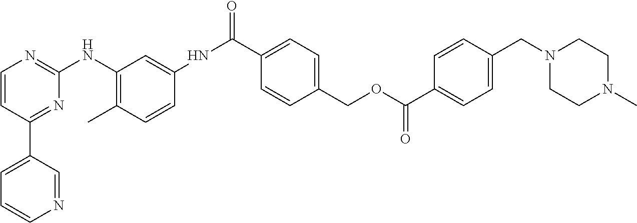 [SMILES: Cc1ccc(NC(=O)c2ccc(COC(=O)c3ccc(CN4CCN(C)CC4)cc3)cc2)cc1Nc1nccc(-c2cccnc2)n1] |

23. Imatinib mesylate of claim 1 having a purity of greater than 99.9% and containing less than about 0.001% of the compound of formula II: 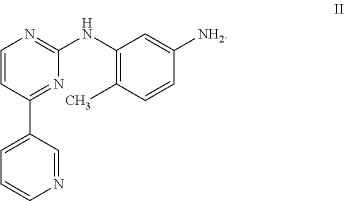 [SMILES: Cc1ccc(N)cc1Nc1nccc(-c2cccnc2)n1]
